(19) United States
(12) Patent Application Publication
(10) Pub. No.: US 2022/0409132 A1
(43) Pub. Date: Dec. 29, 2022

(54) MOUTH GUARD

(71) Applicant: Sang Won YUN, Seoul (KR)

(72) Inventor: Sang Won YUN, Seoul (KR)

(21) Appl. No.: 17/780,157

(22) PCT Filed: Dec. 15, 2020
(86) PCT No.: PCT/KR2020/018352

§ 371 Date: May 26, 2022

(30) Foreign Application Priority Data
Dec. 17, 2019 (KR) 10-2019-0168528

(51) Int. Cl. A61B 5/00; A61B 5/22

(52) U.S. Cl. CPC A61B 5/682; A61B 5/0002; A61B 5/228; A61B 5/746; A61B 2562/0247

(57) ABSTRACT

The present invention relates to a mouth guard having a new structure that protects a user's teeth more effectively, enables a user to feel food texture more effectively when masticating food, and is more firmly fixed to the teeth. The mouth guard according to the present invention comprises a plurality of fixing members 10 made of a hard material and coupled to the teeth 1 to support the teeth 1 and a connecting member 20 made of a synthetic resin material that is softer than the hard material of the fixing member 10 and provided between the fixing members 10 to interconnect the fixing members 10. Since the fixing member 10 is connected to the connection member 20 to be mutually movable by the connection member 20 in a state of being coupled to the user's teeth 1, the mouth guard can effectively protect the user's teeth 1 and can effectively deliver food texture to the teeth 1 when the user chews food. In particular, the mouth guard according to the present invention can prevent sugar and acid generated by a combination of food and saliva from contacting the teeth when eating food, and effectively prevent food debris from being caught between the teeth or between the teeth and gums, thereby preventing various dental diseases such as tooth decay.

### FIELD OF THE INVENTION

The present invention relates to a mouth guard having a new structure that protects a user's teeth more effectively, enables the user to feel food texture more effectively when masticating food, and is more firmly fixed to the teeth.

### BACKGROUND OF THE INVENTION

In general, human teeth are arranged in a U shape on the upper and lower jaw, and teeth in the upper and lower jaw, the upper and lower teeth, are configured to engage with each other.

In addition, when teeth or gum is damaged for various reasons, masticatory movements become very inconvenient.

Meanwhile, among those who experience inconvenience in mastication, some people put a mouth guard on the teeth when chewing food and prevent pain or tooth shaking.

As shown in FIG. 1, the mouth guard has a U-shape corresponding to arrangement of teeth, is formed with a coupling groove 2 that corresponds to the shape of the teeth and is formed on the upper surface or the lower side surface of the mouth guard and is made of a soft synthetic resin material such as silicone.

In this case, the mouth guard coupled to the upper teeth is formed with a coupling groove 2 that is formed on the upper surface of the mouth guard, and the mouth guard coupled to the lower teeth is formed with a coupling groove 2 that is formed on the lower side surface of the mouth guard.

Therefore, when the mouth guard is coupled so that the teeth are inserted into the coupling groove 2, the mouth guard supports the teeth to reduce pain when a user chews food or to support the teeth not to be shaken.

However, there are problems in this mouth guard in that it is difficult to prevent sugar and acid generated by a combination of food and saliva from contacting the teeth, prevent food debris from being caught between the teeth or between the teeth and gums, and prevent various dental diseases such as tooth decay.

In addition, since this mouth guard is made of a synthetic resin material with elasticity, it is difficult for the user to feel food texture when chewing food with the mouth guard, and since a coupling force with the teeth inserted into the coupling groove 2 is weak, the mouth guard may be escaped from the teeth when chewing food.

Therefore, there is a need for a new method to solve these problems.

(Patent Document 1) Korean Registered Patent No. 10-1602539

### DETAILED DESCRITION OF THE INVENTION

### Technical Problems

The present invention has been devised to overcome the problems described above, and objects of the present invention is to provide a mouth guard having a new structure that protects a user's teeth more effectively, that enables the user to feel food texture more effectively when masticating food, and that is more firmly fixed to the teeth.

### SUMMARY OF THE INVENTION

To achieve the objects mentioned above, the present invention provides a mouth guard having a U shape corresponding to arrangement of teeth 1 and formed with a coupling groove 2 that corresponds to a shape of the teeth on an upper surface or a lower side surface thereof, the mouth guard comprising: a plurality of fixing members 10 made of a hard material and coupled to the teeth 1 to support the teeth 1, and a connecting member 20 made of a synthetic resin material that is softer than the hard material of the fixing member 10 and provided between the fixing members 10 to interconnect the fixing members 10.

According to another feature of the present invention, the mouth guard further comprises a penetrating member 12 that is positioned on a bottom surface of an inside of the coupling groove 2 and penetrates the fixing member 10 in a vertical direction, wherein the penetrating member 12 is made of a metal, a synthetic resin material, or a ceramic material having a higher hardness than the fixing member 10 and is in contact with a tooth 1 located in the other side or a penetrating member 12 of the mouthguard that is coupled to the tooth 1 located in the other side to deliver texture of food generated when a user chews the food.

According to another feature of the present invention, the mouth guard further comprises a contact member 11 that is provided on an upper surface or a lower side surface of the mouthguard and extends toward and in close contact with the user's gums, wherein the fixing member 10 is made of a synthetic resin material having elasticity, and the contact member 11 is made of a synthetic resin material that is softer than the fixing member 10.

According to another feature of the present invention, the mouth guard further comprises a pressure sensor 30 provided on a bottom surface of the coupling groove 2 formed in the fixing member 10 at a position corresponding to the user's molar, and a control means 33 configured to receive a signal of the pressure sensor 30 and to be wirelessly communicated with the user's smartphone 34, wherein the control means 33 receives the signal from the pressure sensor 30 and transmits a pressure value with which the user chews food using the molar, to the smartphone 34, wherein the smart phone 34 is provided with a management program 34a, receives the pressure value transmitted from the control means 33, and outputs an alarm when the user chews food with more than a preset pressure value.

### Technical Effects of the Invention

The mouth guard according to the present invention is configured to comprise a plurality of fixing members 10 made of a hard material and coupled to the teeth 1 to support the teeth 1 and a connecting member 20 made of a synthetic resin material that is softer than the hard material of the fixing member 10 and provided between the fixing members 10 to interconnect the fixing members 10. Since the fixing member 10 is connected to the connection member 20 to be mutually movable by the connection member 20 in a state of being coupled to the user's teeth 1, the mouth guard can effectively protect the user's teeth 1 and can effectively deliver food texture to the teeth 1 when the user chews food.

In particular, the mouth guard according to the present invention can prevent sugar and acid generated by a combination of food and saliva from contacting the teeth when eating food, and effectively prevent food debris from being caught between the teeth or between the teeth and gums, thereby preventing various dental diseases such as tooth decay.

### BRIEF DESCRIPTION OF THE DRAWINGS

FIG. 1 is a top view illustrating a conventional mouth guard.

FIG. 2 is a top view illustrating a mouth guard according to the present invention.

FIG. 3 is a top view illustrating an exploded state of the mouth guard according to the present invention.

FIG. 4 is a cross sectional view illustrating a cross section cut along line A-A of FIG. 2.

FIG. 5 is a circuit diagram illustrating a mouth guard according to the present invention.

FIG. 6 is a reference drawing illustrating an action of a mouth guard according to the present invention.

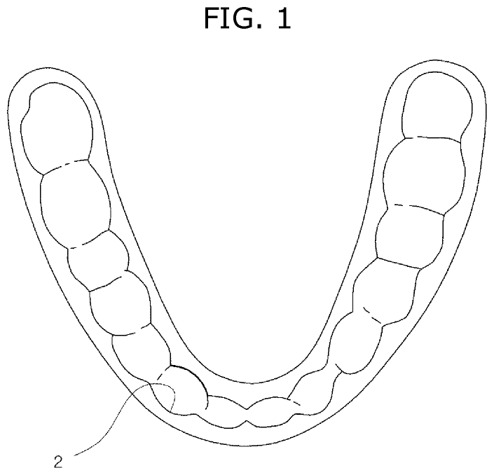

### MODES FOR THE INVENTION

Hereinafter, the present invention will be described in detail in conjunction with accompanying illustrative drawings.

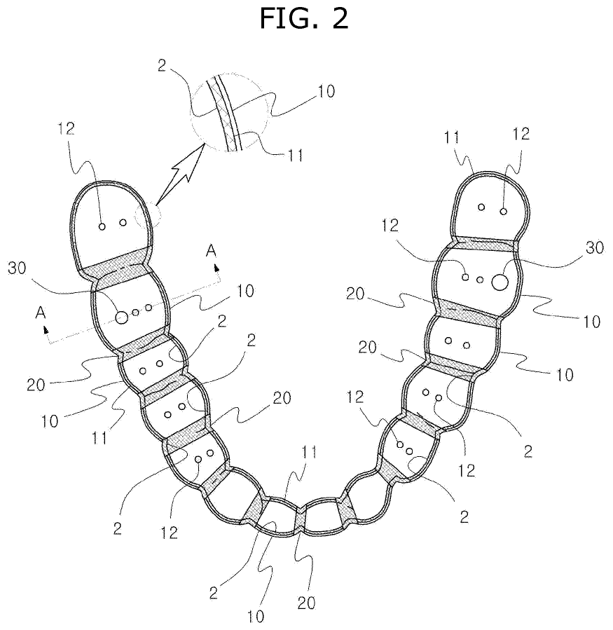

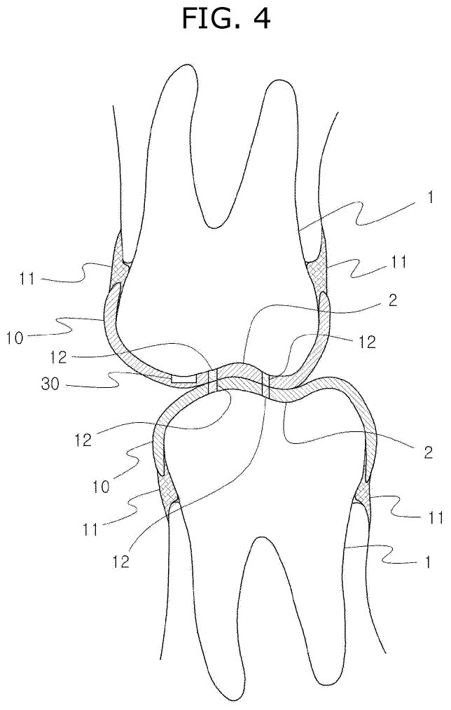

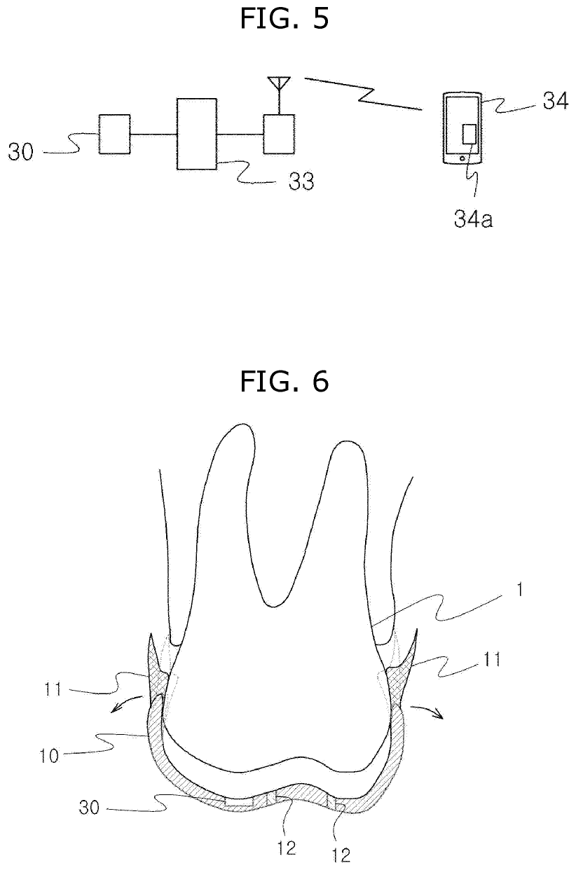

FIGS. 2 to 6 illustrates a mouth guard according to the present invention. The mouth guard is coupled to the upper teeth provided in the upper jaw and the lower teeth provided in the lower jaw, respectively.

In this case, structures in which the mouth guard is configured in a U shape corresponding to arrangement of the teeth 1 and is formed with a coupling groove 2 corresponding to a shape of the teeth 1 are the same as in the prior art.

In addition, the mouth guard according to the present invention is configured to comprise: a plurality of fixing members 10 made of a hard material and coupled to the teeth 1 to support the teeth 1 and a connecting member 20 made of a synthetic resin material that is softer than the hard material of the fixing member 10 and provided between the fixing members 10 to interconnect the fixing members 10.

The fixing member 10 is made of a synthetic resin material that has adequate elasticity and is harmless to the human body. The number of the fixing member 10 corresponds to the number of the tooth 1, and the fixing member 10 is respectively coupled to the user's tooth 1.

Accordingly, as shown in FIG. 6, when the mouth guard is coupled to the tooth 1 or the mouth guard 2 coupled to the tooth is pulled out, the form of the fixing member 10 can be appropriately elastically changed.

In this case, a width of the fixing member 10 in a lateral direction is configured to be smaller than that of the tooth 1, and the fixing member 10 is configured to form a gap in which the connecting member 20 is provided, between the fixing members 10 when the fixing member 10 is coupled to the user's teeth 1.

In addition, on an upper surface and a lower side surface of the mouth guard, that is, the upper surface of the mouth guard coupled to the upper teeth and the lower side surface of the mouth guard coupled to the lower teeth, contact members 11 extending upward and downward to closely adhere to the user's gums 11 are provided.

The contact member 11 is made of a synthetic resin material that is softer than the fixing member 10, such as a silicone material, and the like. The contact member 11 is attached to a front surface and a rear surface of the user's gums and prevents food debris from entering into an inside of the coupling groove 2.

In addition, a penetrating member 12 is provided on a bottom surface of an inside of the coupling groove 2 formed in the fixing member 10 and penetrates the fixing member 10 in a vertical direction.

The penetrating member 12 is made of a metal such as zirconium, gold, or the like a synthetic resin material, or a ceramic material having a higher hardness than the fixing member 10 and is provided to be positioned corresponding to a position where the upper tooth and the lower tooth are engaged when the user chews food.

Accordingly, when the user chews food, the penetrating member 12 becomes in contact with a tooth 1 located in the other side or a penetrating member 12 of the mouthguard that is coupled to the tooth 1 located in the other side and delivers texture of food generated when the user chews the food.

The connection member 20 is made of a soft synthetic resin material such as silicone, and both sides of the connection member 20 are adhesively fixed to the fixing member 10. Accordingly, the connection member 20 performs a function of elastically connecting between the fixing members 10.

In this case, the coupling groove 2 formed in the connecting member 20 is configured to correspond to the shape of the tooth 1.

Furthermore, a pressure sensor 30 is provided on a bottom surface of the coupling groove 2 formed in the fixing member 10 at a position corresponding to the user's molar, and a control means 33 configured to receive a signal of the pressure sensor 30 and to be wirelessly communicated with the user's smartphone 34 is connected to the pressure sensor 30.

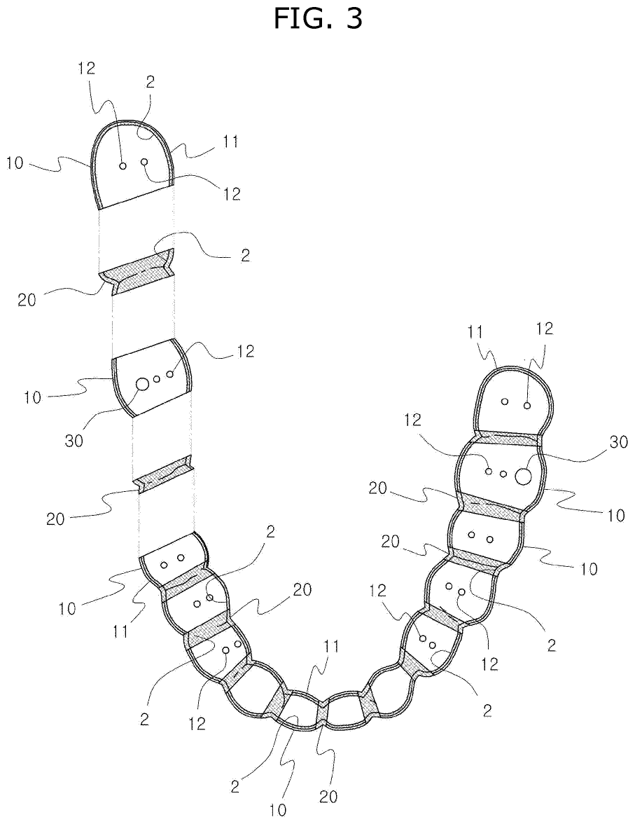

As shown in FIGS. 2 and 3, the pressure sensor 30 is provided in a portion where a force is applied by the molar when the user chews food with the molar and is configured to measure the force applied to the molar when the user chews food.

The control means 33 is communicatively connected to the smart phone 34 through Bluetooth communication and is configured to receive a signal from the pressure sensor 30 and to transmit the pressure value at which the user chews food using the molar, to the smart phone 34.

In this case, the smart phone 34 is provided with a management program 34a.

When the pressure value output from the control means 33 is received, the management program 34a stores the received pressure value and outputs it as a graph, thereby enabling the user to confirm the pressure applied to the molar when the user chews food and assisting the user to correct his or her mastication habit so that the user does not chew food with excessive pressure.

In addition, the management program 34a is configured to receive the pressure value transmitted from the control means 33 and output an alarm when the user chews food with more than a preset pressure value.

Accordingly, it is possible to prevent damage to the user's teeth 1 in advance, which is caused when a user chews food with excessively strong force.

The mouth guard configured as described above is configured to comprise a plurality of fixing members 10 made of a hard material and coupled to the teeth 1 to support the teeth 1 and a connecting member 20 made of a synthetic resin material that is softer than the hard material of the fixing member 10 and provided between the fixing members 10 to interconnect the fixing members 10. Since the fixing members 10 are connected to the connection member 20 to be mutually movable by the connection member 20 in a state of being coupled to the user's teeth 1, the mouth guard can effectively protect the user's teeth 1 and can effectively deliver food texture to the teeth 1 when the user chews food.

In addition, the mouth guard further comprises a penetrating member 12 that is positioned on a bottom surface of an inside of the coupling groove 2 and penetrates the fixing member 10 in a vertical direction, wherein the penetrating member 12 is made of a metal, a synthetic resin material, or a ceramic material having a higher hardness than the fixing member 10 and is in contact with a tooth 1 located in the other side or a penetrating member 12 of the mouthguard that is coupled to the tooth 1 located in the other side to deliver texture of food generated when a user chews the food. Therefore, the user can feel food texture more effectively.

Furthermore, the mouth guard further comprises a contact member 11 that is provided on an upper surface or a lower side surface of the mouthguard and extends toward and in close contact with the user's gums, wherein the fixing member 10 is made of a synthetic resin material having elasticity, and the contact member 11 is made of a synthetic resin material that is softer than the fixing member 10. Since the contact member 11 prevents food debris from entering into an inside of the coupling groove 2, the mouth guard can prevent sugar and acid generated by a combination of food and saliva from contacting the teeth when eating food and effectively prevent food debris from being caught between the teeth or between the teeth and gums, thereby preventing various dental diseases such as tooth decay.

In addition, the mouth guard further comprises a pressure sensor 30 provided on a bottom surface of the coupling groove 2 formed in the fixing member 10 at a position corresponding to the user's molar, and a control means 33 configured to receive a signal of the pressure sensor 30 and to be wirelessly communicated with the user's smartphone 34, wherein the control means 33 receives the signal from the pressure sensor 30 and transmits a pressure value with which the user chews food using the molar, to the smartphone 34, wherein the smart phone 34 is provided with a management program 34a, receives the pressure value transmitted from the control means 33, and outputs an alarm when the user chews food with more than a preset pressure value. Therefore, the mouth guard can protect the teeth 1 more effectively.

In the present embodiment, the mouth guard has been illustrated as being coupled to the upper teeth provided in the upper jaw and the lower teeth provided in the lower jaw at the same time but is not limited thereto. The mouth guard according to the present invention may be coupled only to any one of the upper teeth and the lower teeth.

What is claimed is:

1. A mouth guard having a U shape corresponding to arrangement of teeth 1 and formed with a coupling groove 2 that corresponds to a shape of the teeth 1 on an upper surface or a lower side surface thereof, the mouth guard comprising:
a plurality of fixing members 10 made of a hard material and coupled to the teeth 1 to support the teeth 1, and
a connecting member 20 made of a synthetic resin material that is softer than the hard material of the fixing member 10 and provided between the fixing members 10 to interconnect the fixing members 10.

2. The mouth guard of claim 1, further comprising a penetrating member 12 that is positioned on a bottom surface of an inside of the coupling groove 2 and penetrates the fixing member 10 in a vertical direction,
wherein the penetrating member 12 is made of a metal, a synthetic resin material, or a ceramic material that has a higher hardness than the fixing member 10 and is in contact with a tooth 1 located in the other side or a penetrating member 12 of the mouth guard that is coupled to the tooth 1 located in the other side to deliver food texture generated when a user chews the food.

3. The mouth guard of claim 1, further comprising a contact member 11 that is provided on an upper surface or a lower side surface of the mouthguard and extends toward and in close contact with the user's gums,
wherein the fixing member 10 is made of a synthetic resin material having elasticity, and the contact member 11 is made of a synthetic resin material that is softer than the fixing member 10.

4. The mouth guard of claim 1, further comprising:
a pressure sensor 30 provided on a bottom surface of the coupling groove 2 formed in the fixing member 10 at a position corresponding to the user's molar, and
a control means 33 configured to receive a signal of the pressure sensor 30 and to be wirelessly communicated with the user's smartphone 34,
wherein the control means 33 receives the signal from the pressure sensor 30 and transmits a pressure value with which the user chews food using the molar, to the smartphone 34,
wherein the smart phone 34 is provided with a management program 34a, receives the pressure value transmitted from the control means 33, and outputs an alarm when the user chews food with more than a preset pressure value.
